(19) United States
(12) Patent Application Publication
(10) Pub. No.: US 2025/0009952 A1
(43) Pub. Date: Jan. 9, 2025

(54) APPARATUS AND METHOD FOR REMOVING EXCESS TRANSTHYRETIN PROTEIN FROM BLOODSTREAM TO TREAT ATTR AMYLOIDOSIS

(71) Applicant: The FairfaxWood Amyloid Initiative, Boca Raton, FL (US)

(72) Inventor: John A. Wood, Boca Raton, FL (US)

(21) Appl. No.: 18/764,739

(22) Filed: Jul. 5, 2024

Related U.S. Application Data
(60) Provisional application No. 63/525,034, filed on Jul. 5, 2023.

(51) Int. Cl. A61M 1/36

(52) U.S. Cl. CPC A61M 1/3618; A61M 1/3603; A61M 1/36222; A61M 1/3626; A61M 2202/0014; A61M 2202/0021; A61M 2202/0445; A61M 2205/0244; A61M 2205/0272; A61M 2205/10; A61M 2205/121; A61M 2205/3331

(57) ABSTRACT

An apparatus for treating a patient suffering from acquired Transthyretin Amyloidosis includes a blood dialysis circuit with a pump for pumping the patient's blood that has been pretreated with magnetic nanoparticles through the dialysis circuit, and a transthyretin amyloid (TTR) protein separation component downstream of the pump and structured and disposed to separate the TTR proteins from the patient's blood by magnetic attraction as a result of the magnetic nanoparticles infiltrating the TTR proteins prior to circulation through the circuit. The circuit further includes a pressure gauge and a downstream end connection to the patient's venous blood flow for returning the patient's blood to the patient after removal of the TTR proteins.

### BACKGROUND OF THE INVENTION

This non-provisional patent application is based on provisional patent application serial no. 63/525,034 filed on Jul. 5, 2023.

### Field of the Invention

The present invention relates to a device for removing excess TTR proteins in a patient's blood for treating Amyloidosis and, more particularly, for a system and device for removing TTR proteins from a patient's blood by magnetic attraction.

### DESCRIPTION OF THE RELATED ART

Amyloidosis is a blood disease that occurs when a protein called Amyloid builds up in organs, joints, vertebrae and nerve bundles. More particularly, Amyloidosis breaks down two types of protein in the blood into minute fibrils. These fibrils deposit in various organs, such as the heart, kidneys, liver, spleen, nervous system and digestive track, as well as joints and vertebrae. There are two types of Amyloidosis, Light Chain and Transthyretin. Each of these two types are further divided into two subgroups, either acquired or hereditary. Three of these types and subtypes are treatable with the exception of the acquired Transthyretin (ATTR) Amyloidosis subgroup.

The present invention proposes use of FDA approved kidney dialysis machines to reduce the quantity of Transthyretin in the blood to slow the development of this fatal condition. The full operational and safety features of the FDA approved kidney dialysis machines are maintained. The invention requires only the interchange of a single machine part of the kidney dialysis machine.

### SUMMARY OF THE INVENTION

The present invention removes excess TTR proteins by magnetic attraction and collects them in a plastic pouch for discard. This is accomplished with use of FDA approved kidney dialysis machines that are modified by removing the cartridge filter of the kidney dialysis machines and replacing the filter with a similarly attached device that removes the excess TTR proteins by magnetic attraction. This requires pre-treatment of the patient to infuse magnetic nanoparticles into the blood to infiltrate the proteins. A non-magnetic tray is attached to the kidney dialysis machine in the same manner as the cartridge filter. A magnetic pad rests on the tray, and the collection pouch rests on the magnetic pad. Arterial and venous blood catheter connections are made from the patient to the pouch. As blood is pumped through the pouch, the magnetic attraction will draw the now magnetized proteins to the bottom surface of the pouch. When the treatment is complete, the blood flow is stopped, and the pouch is removed and discarded.

A post-treatment of iron supplement to replace lost ferritins is also required. Duration and spacing of the treatments will be determined by the individual's physician.

### BRIEF DESCRIPTION OF THE DRAWINGS

For a fuller understanding of the nature of the present invention, reference should be had to the following detailed description taken in connection with the accompanying drawings in which:

FIG. 1 illustrates a typical kidney dialysis circuit which includes a dialyzer filter cartridge in the blood flow circuit;

FIG. 2 is an isolated exploded view, showing a replacement component of the present invention that replaces the dialyzer filter cartridge of the kidney dialysis machine, and the replacement component including a plastic pouch, a magnetic pad, and a non-magnetic tray which is specifically structured for attachment to the existing filter clamp of the kidney dialysis machine;

FIG. 3A is a side elevational view of the blow molded plastic pouch;

FIG. 3B is a cross-sectional view taken along the line indicated by the arrows A-A in FIG. 3A;

FIG. 3C is an isolated detail taken from the area indicated as “1” in FIG. 3A;

FIG. 4 is a perspective view generally illustrating a typical FDA approved dialysis machine;

FIG. 5 is an exploded view showing the filter clamp and disposable cartridge filter of a typical FDA approved kidney dialysis machine; and

FIG. 6 illustrates a blood dialysis circuit according to the present invention and including the TTR protein removal component that is structured and disposed for removing TTR proteins from the patient's blood by magnetic attraction as the patient's blood flows through the circuit.

Like reference numerals refer to like parts throughout the several views of the drawings.

### DETAILED DESCRIPTION OF THE PREFERRED EMBODIMENT

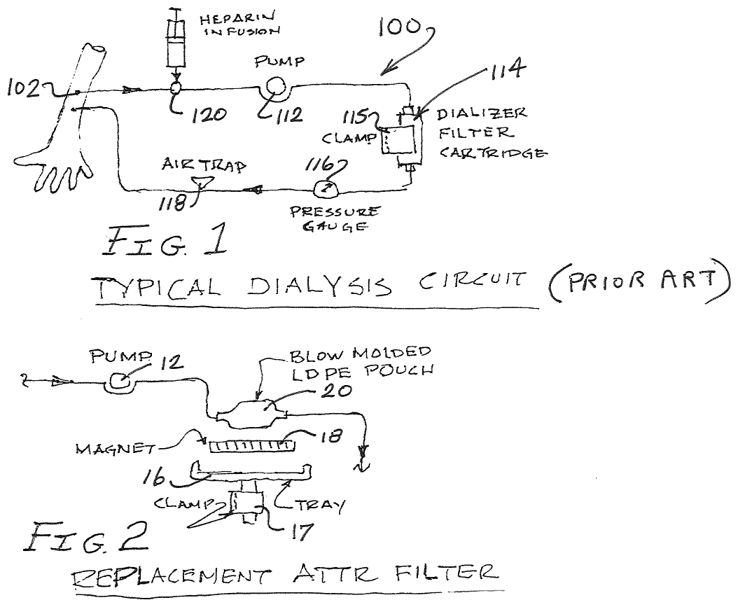

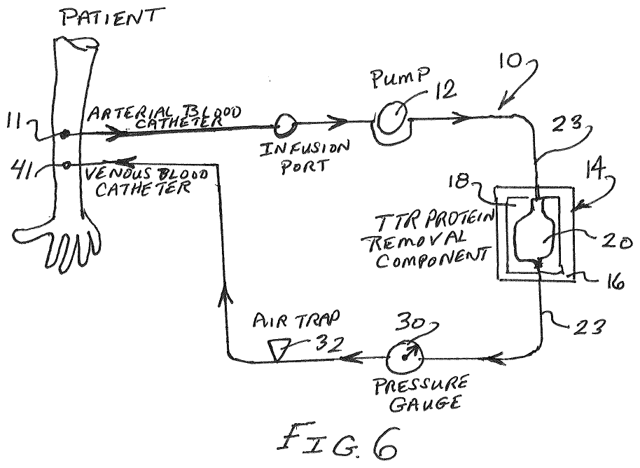

Referring initially to FIG. 1, a dialysis circuit of a typical kidney dialysis machine in the prior art is shown and is generally indicated as 100. The kidney dialysis machine 110 (see FIG. 4) takes blood from the patient and pumps it through a filter 114 to clear body debris. Safety controls, including for air exclusion, temperature and pressure are included. The filter is a removable cartridge with an inlet and outlet port. More particularly, the dialysis circuit of a typical kidney dialysis machine, as shown in FIG. 1, includes a pump 112 for pumping blood through the circuit. The pump 112 is in fluid flow connection to the patient's arterial bloodstream at a first intravenous connection point 102. Downstream of the pump 112 there is a dialyzer filter cartridge 114 that is held in place by a clamp 115. After body debris is removed in the filter cartridge, the blood continues to flow through the circuit, and through a pressure gauge 116 and past an air trap 118 that prevents air from being reintroduced into the patient's bloodstream. The opposite end of the circuit is connected in fluid flow connection to the patient's venous bloodstream via an intravenous connection. The dialysis circuit may also include an infusion port 120 for injecting various drugs or treatment substances, such as HEPARIN.

Referring to FIG. 6, the blood dialysis circuit of the present invention is shown and is generally indicated as 10. The blood dialysis circuit 10 includes a pump 12 in fluid flow connection with the patient's arterial bloodstream via an arterial blood catheter 11 much like in the dialysis circuit of FIG. 1. As previously noted, the dialyzer filter cartridge and clamp in the dialysis circuit for a kidney dialysis machine, as seen in FIG. 1, is replaced with a TTR protein removal component 14. The dialysis circuit 10 further includes a pressure gauge 30 and an air trap 32 to remove air prior to delivering the cleansed blood back into the patient's bloodstream.

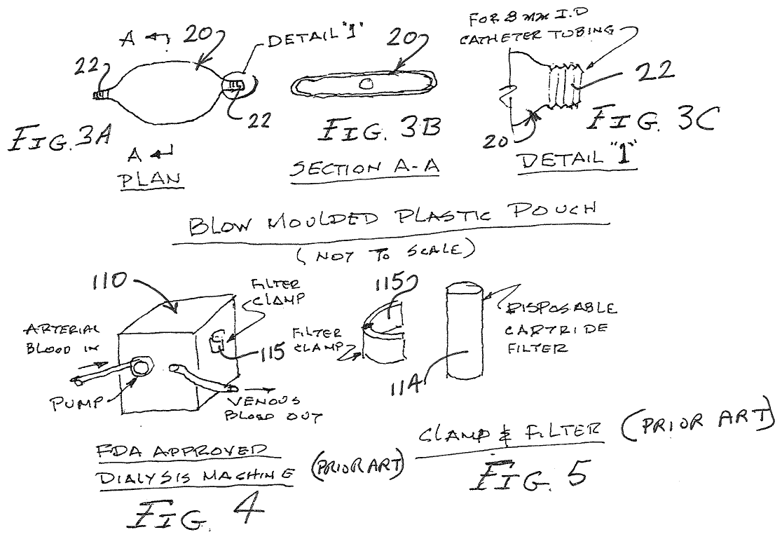

Referring to FIG. 2, the present invention proposes to remove the filter cartridge 114 of the FDA approved kidney dialysis machine shown in FIGS. 1 and 4 and replace the filter cartridge with a similarly attached device to remove the excess TTR proteins. More particularly, the present invention provides a TTR protein separator device 14 that works by magnetic attraction to collect the TTR proteins in a plastic blow molded pouch 20 for discard. The TTR protein removal device 14 consists of a non-magnetic tray 16 attached to the dialysis machine in the same manner as the cartridge filter using a clamp 17 (see FIG. 2). The TTR protein removal device 14 further includes a magnetic pad 18 that rests on the tray 16 and the blow molded plastic collection pouch 20 which rests on top of the magnetic pad 18. In operation, arterial 11 and venous 41 blood catheter connections are made from the patient to the dialysis circuit 10 including the pouch 20. As seen in FIGS. 3A-3C, the opposite ends of the pouch have thread connect ports 22 for connection to 8 mm I.D. catheter tubing 23 that carries the patient's blood through the blood dialysis circuit. In order to make the TTR proteins magnetic, the patient's blood must first be pre-treated with magnetic nanoparticles that are sized to infiltrate the proteins. As blood is pumped through the pouch 20 in the dialysis circuit 10, the magnetic attraction created by the magnetic pad 18 will draw the now magnetized TTR proteins to the bottom surface of the pouch 20. When the treatment is complete, blood flow is stopped, and the pouch 20 is removed and discarded. Pre-treatment of infusion of the magnetic nanoparticles into the bloodstream along with use of oxygen to elevate blood oxygen level near one hundred percent (to demagnetize hemoglobin) is required. A post-treatment of iron supplement to replace lost ferritins is also required. Duration and spacing of the treatments will be determined by the individual's physician.

Since many modifications, variations and changes in detail can be made to the described embodiments of the invention, it is intended that all matters in the foregoing description and shown in the accompanying drawings be interpreted as illustrative and not in a limiting sense. Thus, the scope of the invention should be determined by the appended claims and their legal equivalents.

What is claimed is:

1. An apparatus for treating a patient suffering from acquired Transthyretin Amyloidosis, the apparatus comprising:
a blood dialysis circuit comprising:
a pump in fluid flow connection to the patient's arterial blood stream for pumping blood through the dialysis circuit;
a transthyretin amyloid protein separation component downstream of the pump in the circuit, and the transthyretin amyloid protein separation component structured and disposed for separating transthyretin amyloid proteins from the patient's blood that has been pretreated with an infusion of magnetic nanoparticles, the transthyretin amyloid protein separation component being structured and 11 disposed to separate the transthyretin amyloid proteins from the patient's blood by 12 magnetic attraction;
at least one pressure gauge; and
the dialysis circuit having a downstream end connected to the patient's venous blood flow for returning the patient's blood to the patient after removal of the transthyretin amyloid proteins.

2. The apparatus as recited in claim 1 wherein the transthyretin amyloid protein separation component includes:
a magnetic pad; and
a pouch including ports at opposite ends, including a first port for receiving the patient's blood from the pump and a second, opposite port for releasing the blood back into the blood dialysis circuit for eventual return to the patient's venous blood flow, and wherein the transthyretin amyloid proteins are separated from the blood in the pouch by magnetic attraction to the magnetic pad below the pouch.

3. The apparatus as recited in claim 2 further comprising:
a non-magnetic tray fixed to the blood dialysis circuit and structured and disposed for supporting the magnetic pad and the pouch on a top surface of the non-magnetic tray.

4. The apparatus as recited in claim 1 further including an air trap for removing air prior to delivering the patient's cleansed blood after removal of the transthyretin amyloid proteins back into the patient's bloodstream.

5. A method for treating a patient suffering from acquired Transthyretin Amyloidosis, the method comprising the steps of:
pretreating the patient's blood with an infusion of magnetic nanoparticles that infiltrate transthyretin amyloid proteins in the patient's bloodstream;
directing the patient's arterial blood flow through a blood dialysis circuit using a pump and an arterial blood catheter connection to the patient;
separating the transthyretin amyloid proteins that have the magnetic nanoparticles infiltrated therein by magnetic attraction; and
directing the patient's blood after removal of the transthyretin amyloid proteins therefrom through the circuit and to the patient's venous bloodstream via a venous blood catheter connection at the opposite end of the blood dialysis circuit.
